(19) United States
(12) Patent Application Publication
(10) Pub. No.: US 2009/0270891 A1
(43) Pub. Date: Oct. 29, 2009

(54) Balanced ultrasonic curved blade

(76) Inventor: Jean Michael Beaupre, Alexandria, KY (US)

Correspondence Address: Jean M. Beaupre, Suite 412, 4480 Lake Forest Dr., Cincinnati, OH, 45242, US

(21) Appl. No.: 12/386,465

(22) Filed: Apr. 17, 2009

Related U.S. Application Data
(60) Provisional application No. 61/124,642, filed on Apr. 18, 2008.

(51) Int. Cl. A61B 17/32

(52) U.S. Cl. 606/169

(57) ABSTRACT

Methods and devices that provide reduced transverse motion in a curved ultrasonic blade and/or ultrasonic surgical instrument with functional asymmetries. An ultrasonic blade in accordance with embodiments of the present invention includes a curved functional portion of an ultrasonic blade, wherein the center of mass of the curved functional portion lies on the mid-line of a waveguide delivering ultrasonic energy to the blade. Balancing in accordance with embodiments of the present invention, using placement of the center of mass of the curved portion of the blade appropriately, provides blade balance in a proximal portion of the blade, without reduction of mass and inherent stress increase proximal to the end-effector.

This application hereby claims the priority of U.S. Provisional Application 61/124,642 filed on Apr. 18, 2008. U.S. Provisional Application 61/124,642 is incorporated by reference.

### FIELD OF THE INVENTION

The present invention relates, in general, to ultrasonic devices and, more particularly, to methods and devices that provide curved blades with reduced undesired laterial and torsion motion.

### BACKGROUND OF THE INVENTION

The fields of ultrasonics and stress wave propagation encompass applications ranging from non-destructive testing in materials science, to beer packaging in high-volume manufacturing. Diagnostic ultrasound uses low-intensity energy in the 0.1-to-20-MHz region to determine pathological conditions or states by imaging. Therapeutic ultrasound produces a desired bio-effect, and can be divided further into two regimes, one in the region of 20 kHz to 200 kHz, sometimes called low-frequency ultrasound, and the other in the region from 0.2 to 10 MHz, where the wavelengths are relatively small, so focused ultrasound can be used for therapy. At high intensities of energy, this application is referred to as HIFU for High Intensity Focused Ultrasound.

Examples of therapeutic ultrasound applications include HIFU for tumor ablation and lithotripsy, phacoemulsification, thrombolysis, liposuction, neural surgery and the use of ultrasonic scalpels for cutting and coagulation. In low-frequency ultrasound, direct contact of an ultrasonically active end-effector or surgical instrument delivers ultrasonic energy to tissue, creating bio-effects. Specifically, the instrument produces heat to coagulate and cut tissue, and cavitation to help dissect tissue planes. Other bio-effects include: ablation, accelerated bone healing and increased skin permeability for transdermal drug delivery.

Ultrasonic medical devices are used for the safe and effective treatment of many medical conditions. Ultrasonic surgical instruments are advantageous because they may be used to cut and/or coagulate organic tissue using energy, in the form of mechanical vibrations, transmitted to a surgical end-effector at ultrasonic frequencies. Ultrasonic vibrations, when transmitted to organic tissue at suitable energy levels and using a suitable end-effector, may be used to cut, dissect, or cauterize tissue.

Ultrasonic vibration is induced in the surgical end-effector by, for example, electrically exciting a transducer which may be constructed of one or more piezoelectric or magnetostrictive elements in the instrument hand piece. Vibrations generated by the transducer section are transmitted to the surgical end-effector via an ultrasonic waveguide extending from the transducer section to the surgical end-effector. The waveguide/end-effector combinations are typically designed to resonate at the same frequency as the transducer. Therefore, when an end-effector is attached to a transducer the overall system frequency is still the same frequency as the transducer itself.

At the tip of the end-effector, ultrasonic energy is delivered to tissue to produce several effects. Effects include the basic gross conversion of mechanical energy to both frictional heat at the blade-tissue interface, and bulk heating due to viscoelastic losses within the tissue. In addition, there may be the ultrasonically induced mechanical mechanisms of cavitation, microstreaming, jet formation, and other mechanisms.

Ultrasonic surgical instruments utilizing solid core technology are particularly advantageous because of the amount of ultrasonic energy that may be transmitted from the ultrasonic transducer through a solid waveguide to the active portion of the end-effector, typically designated as a blade. Such instruments are particularly suited for use in minimally invasive procedures, such as endoscopic or laparoscopic procedures, wherein the end-effector is passed through a trocar to reach the surgical site.

Solid core ultrasonic surgical instruments may be divided into two types, single element end-effector devices and multiple-element end-effector. Single element end-effector devices include instruments such as scalpels, and ball coagulators, see, for example, U.S. Pat. No. 5,263,957. Multiple element end-effectors include those illustrated in devices such as ultrasonic shears, for example, those disclosed in U.S. Pat. Nos. 5,322,055 and 5,893,835 provide an improved ultrasonic surgical instrument for cutting/coagulating tissue, particularly loose and unsupported tissue. The ultrasonic blade in a multiple-element end-effector is employed in conjunction with a clamp for applying a compressive or biasing force to the tissue. Clamping the tissue against the blade provides faster and better controlled coagulation and cutting of the tissue.

In an ultrasonic device running at resonance in primarily a longitudinal mode, the longitudinal ultrasonic motion, d, behaves as a simple sinusoid at the resonant frequency as given by:

d=A sin(ω·t)

where: ω=the radian frequency, which equals (2·π) multiplied by the cyclic frequency, f; t is time; and A=the zero-to-peak amplitude.

The longitudinal excursion is defined as the peak-to-peak amplitude, which is twice the amplitude of the sine wave, mathematically expressed as 2·A.

An ultrasonic waveguide and blade in perfect balance over its entire length will vibrate longitudinally according to this simple harmonic motion. Unfortunately, ultrasonic blades are not typically in perfect balance. For example, blades useful for medical applications may incorporate asymmetrical features, including but not limited to curves, that cause blade imbalances. U.S. Pat. Nos. 6,283,981 and 6,328,751 and U.S. patent application Ser. No. 11/261,243 disclose methods and designs for ultrasonic instruments that are transverse balanced.

Furthermore, it has been found that ultrasonic devices with asymmetrical motion as disclose in U.S. patent application Ser. No. 11/411,731 can provide benefits beyond longitudinal motion devices.

However, current known methods of producing asymmetrical motion in ultrasonic devices cannot be combined with balanced asymmetrical ultrasonic devices without producing a harmonic axial torsion distortion in the waveguide. This is particularly true when the plane of asymmetrical motion is non-parallel to the plane of end effector curvature.

### SUMMARY OF THE INVENTION

The present invention is directed to methods and devices that provide reduced transverse and axial torsion motion in a curved ultrasonic blade while simultaneously providing ultrasonic clamped cutting using asymmetrical motion with an asymmetrical device. An ultrasonic blade in accordance with embodiments of the present invention includes a curved functional portion of an ultrasonic blade. Said ultrasonic blade also includes a plurality of notches proximal to the end effector, configured to reduce axial torsion motion. Reducing transverse motion in the proximal portion of the blade in accordance with embodiments of the present invention may be accomplished by placement of the center of mass of the function portion of the blade appropriately or by inclusion of features proximal to the functional portion. Creating asymmetrical motion in the functional portion and reducing axial torsion motion in the proximal portion of the blade in accordance with embodiments of the present invention may be accomplished by including asymmetrical features proximal to the functional portion.

Embodiments of ultrasonic surgical devices in accordance with the present invention include an elongated waveguide configured to transmit ultrasonic energy. The elongated waveguide has a center-line extending through the center of mass. An end-effector is provided at the distal end of the waveguide, and includes a curved portion having a positive curvature. The positive curvature of the curved portion produces an offset of the center of mass of the curved portion. An anti-curved portion is positioned between the elongated waveguide and the curved portion, the anti-curve having a negative curvature, the negative curvature configured to correct the offset of the center of mass of the curved portion, thereby substantially reducing transverse motion in the waveguide of the ultrasonic surgical device. A plurality of asymmetrical notches with a center of mass on the center line are included proximal to the end-effector, the asymmetrical notches configured to produce asymmetrical motion in the end effector and reduce axial torsion motion.

Other embodiments include using an end effector with center of mass not on the centerline, a plurality of transverse balance asymmetries, and a plurality of asymmetrical features proximal to the end-effector to produce asymmetrical motion in the end effector and reduce axial torsion. Further embodiments include a clamp arm configured to opposably clamp tissue against the curved portion, wherein the clamp arm is actuatably movable from an open position to a clamped position.

Methods of balancing ultrasonic systems in accordance with embodiments of the present invention involve determining a center-line that extends through the center of mass of a first portion of an ultrasonic system. A center of mass of a second portion of the ultrasonic system is determined, the second portion comprising a functional asymmetry. The center of mass of the second portion is located about the center-line of the first portion using a curved portion of the ultrasonic system, the curved portion positioned between the first portion and the second portion. A center of mass of a third portion of the ultrasonic system is determined, the third portion comprising of non-functional asymmetries. The center of mass of the third portion is located about the center-line of the first portion.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

The features of the invention may be set forth with particularity in the appended claims. The invention itself, however, both as to organization and methods of operation, together with further objects and advantages thereof, may best be understood by reference to the following description, taken in conjunction with the accompanying drawings in which:

FIG. 1 is a perspective view of a balanced ultrasonic blade having an asymmetry in accordance with prior art;

FIG. 2 is a side view of a balanced ultrasonic blade having asymmetry in accordance with prior art, showing the center of gravity with relation to the center line;

FIG. 3 is a top view of a balanced ultrasonic blade having asymmetry in accordance with prior art, showing the center of gravity with relation to the center line;

FIG. 4 is a side view of a blade producing asymmetrical motion in accordance with prior art, showing the motion components;

FIG. 5 is a side view of a blade producing asymmetrical motion in accordance with prior art while at maximum excursion;

FIG. 6A is a top view of a blade combining features of FIG. 1 and features of FIG. 4 as described in prior art;

FIG. 6B is a side view of a blade combining features of FIG. 1 and features of FIG. 4 as described in prior art;

FIG. 7 is a perspective view of a blade combining features of FIG. 1 and features of FIG. 4 as described in prior art, showing the axial torsion motion;

FIG. 8A is a top view of a blade combining features of FIG. 1 and asymmetrical notches configured to produce asymmetrical motion in the end effector and reduce axial torsion motion;

FIG. 8B is a side view of a blade combining features of FIG. 1 and asymmetrical notches configured to produce asymmetrical motion in the end effector and reduce axial torsion motion;

FIG. 9 is a perspective view of a blade combining features of FIG. 1 and asymmetrical notches configured to produce asymmetrical motion in the end effector and reduce axial torsion motions.

FIG. 10 is a perspective view of a blade that is configured to operate accordance with the present invention and in clamping cooperation with a clamp arm.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF THE INVENTION

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

Considerable effort has been directed at correcting imbalances inherent in curved ultrasonic blades and ultrasonic devices that are not symmetric about their longitudinal axis. Descriptions of methods to correct ultrasonic blade imbalances are described in U.S. Pat. Nos. 6,283,981; 6,328,751; 6,660,017; 6,325,811; 6,432,118; and 6,773,444, and U.S. patent application Ser. Nos. 11/348,911 and 11/411,731 which are hereby incorporated herein by reference. Although balancing of ultrasonic blades has greatly expanded the possibilities of blade design, balancing using the methodologies described in U.S. Pat. Nos. 6,283,981; 6,328,751; 6,660,017; 6,325,811; 6,432,118; and 6,773,444 as well as U.S. patent application Ser. No. 11/348,911 and Ser. No. 11/411,731 describe balancing of ultrasonic blades that are excited solely by longitudinal motion.

U.S. patent application Ser. No. 11/261,243 discloses methods and designs for exciting an ultrasonic blade with a symmetrical end effector with non-longitudinal motion. However, exciting an asymmetrical end effector with non-longitudinal motion may result in creating a tertiary motion within the end-effector and waveguide. This tertiary motion is comprised of axial torsion motion and additional transverse motion. This tertiary motion may provide benefits for the function of the end effector. However, transverse and axial torsion motion are known to create heat, noise, and reduced component life within the waveguide and support components. These motions may also propagate proximal to the waveguide and damage the ultrasonic power source, such as a transducer.

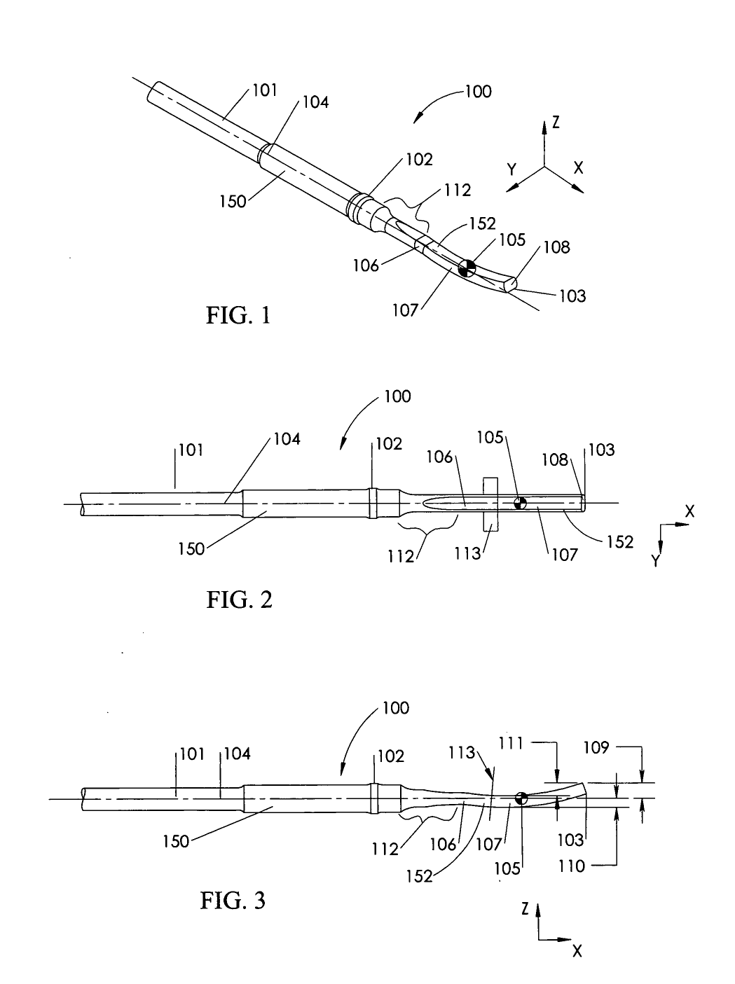

Referring now prior art shown in FIG. 1, a perspective view of an ultrasonic surgical instrument 100 is illustrated, including a waveguide 150 and a blade 152. As described in U.S. patent application Ser. No. 11/411,731, the ultrasonic surgical instrument 100 includes a curved treatment portion 107 for use in medical procedures to, for example, dissect or cut living organic tissue. A distal flat working surface 108 is illustrated as terminating the curved treatment portion 107, and may be used for spot coagulation, plane dissection, or other surgical procedure.

A center of mass 105 of the curved treatment portion 107 is located on a central axis 104 of the waveguide 150. The central axis 104 may be defined as the center-line of a circularly symmetric blade extending along the longitudinal direction, or a line extending in the primary vibrational-mode direction and passing through the center of mass, for blades that are not circularly symmetric. The center of mass 105 is illustrated in FIG. 1 as about 0.01 inches transversely from the central axis 104, and may be about 0.0003 inches transversely from the central axis 104.

The ultrasonic surgical instrument 100 is illustrated in FIG. 1 as extending from a proximal anti-node 101 to a distal anti-node 103, with a distal node 102 approximately half way between the proximal anti-node 101 and the distal anti-node 103. An amplifier 112 may be included to amplify the excursion of the blade. The amplifier 112 may provide about a multiple of 2 amplification (about a one-half reduction of cross-sectional area.) An anti-curve 106 may be positioned between the distal node 102 and the curved treatment portion 107, to position the center of mass 105 at or near the central axis 104, thereby providing reduction of transverse motion in the waveguide 150 in accordance with the present invention. The anti-curve 106 and the curved treatment portion 107 may be used in combination as a functional portion of the ultrasonic surgical instrument 100 in particular embodiments of the present invention. In other embodiments, the anti-curve 106 may be provided proximal to the functional portion of the ultrasonic surgical instrument 100. In the particular embodiment illustrated in FIGS. 1 through 3, the anti-curve 106 and the curved treatment portion 107 are both part of the functional portion of the blade. The anti-curve 106 is illustrated in FIG. 1 as about 0.053 inches to about 0.061 inches in length, and may be about 0.015λ to about 0.018λ in some alternate embodiments.

In the particular embodiment illustrated in FIG. 1, the cross sections of the curved treatment portion 107 and the waveguide 150 are symmetrical. The deflection of the curved treatment portion 107 of the ultrasonic surgical instrument 100 is substantial, in order to create an out and around shape to aid in medical surgical procedures, and to allow passage through a trocar or endoscopic surgical port (not shown.) For example, the curvature of the curved treatment portion 107 is illustrated as having a continuous or varying arc of about 15 to 30 degrees that may be accomplished, for example, using a radius of curvature of about 1.2 inches over a length of about 0.6 inches. In the particular embodiment illustrated in FIGS. 1 through 3, the radius of curvature is illustrated as 1.192 inches through an arc of about 27.22 degrees. The radius of curvature for top and bottom surfaces of the curved treatment portion 107 may be different. For example, the bottom surface of the curved treatment portion 107 may have a radius of curvature of about 1.22 inches, while the top surface of the curved treatment portion 107 may have a radius of curvature of about 1.163 inches.

The ultrasonic surgical instrument 100 is preferably made from a solid core shaft constructed of material which propagates ultrasonic energy, such as a titanium alloy (i.e., Ti-6Al-4V) or an aluminum alloy. It will be recognized that the ultrasonic surgical instrument 100 may be fabricated from any other suitable material. It is also contemplated that the ultrasonic surgical instrument 100 may have a surface treatment to improve the delivery of energy and desired tissue effect. For example, the ultrasonic surgical instrument 100 may be micro-finished, coated, plated, etched, grit-blasted, roughened or scored to enhance coagulation and cutting of tissue and/or reduce adherence of tissue and blood. Additionally, the ultrasonic surgical instrument 100 may be sharpened or shaped to enhance its characteristics. For example, a portion of the curved treatment portion 107 may be shaped, sharpened, or have some other desired shape.

FIGS. 2 and 3 are top and side views respectively of the ultrasonic surgical instrument 100 illustrated in FIG. 1, illustrating the three dimensional positioning of the center of mass 105 relative to the central axis 104. In the particular example illustrated in FIGS. 1 through 3, the anti-curve 106 is illustrated as angling the curved treatment portion 107 about 6 degrees to about 12 degrees, and more particularly about 8.13 degrees, to position the center of mass 105 about the central axis 104, thereby reducing undesired transverse motion in the waveguide 150.

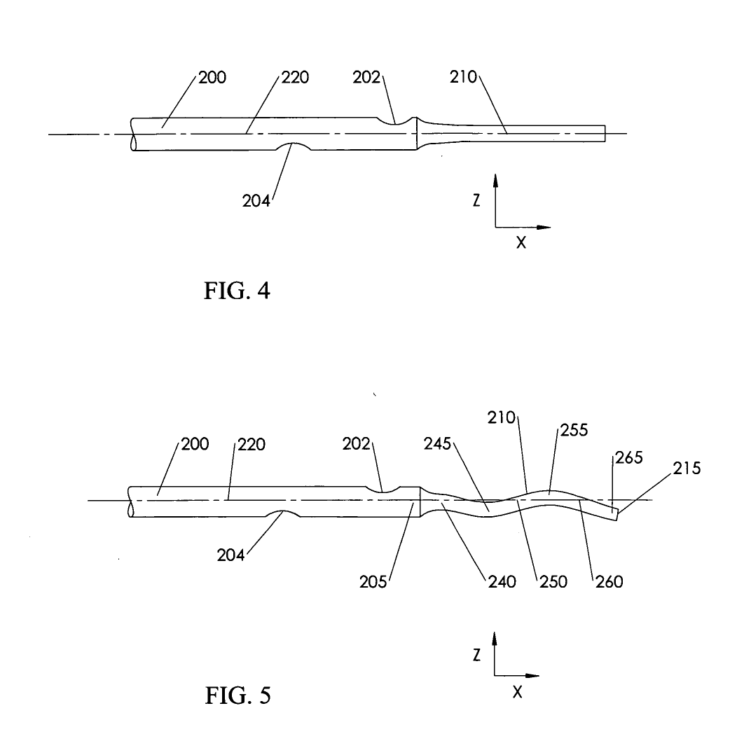

FIG. 4 is a magnified plan view of the waveguide 200 and cutting blade 210, where the cutting blade 210 is illustrated at rest. Notches 202 and 204 induce lateral motion in cutting blade 210 but not waveguide 200. FIG. 5 is a magnified plan view of waveguide 200 and cutting blade 210 of FIG. 4, where cutting blade 210 is illustrated at an exaggerated excursion in an expansion phase of ultrasonic motion. The x-direction is defined as parallel to the longitudinal axis 220 while the y-direction is defined as perpendicular to the longitudinal axis 220 and shown as the vertical axis in FIGS. 4 and 5. The ultrasonic motion of the cutting blade 210 is seen in FIG. 5 to have concurrent y-direction motion and x-direction motion. The x-direction motion in the waveguide 200 and cutting blade 210 may have a node 205 and an anti-node 215. The concurrent y-direction motion (vertical axis) may have nodes 240, 250 and 260, and anti-nodes 245, 255, and 265.

The ultrasonic surgical instrument 100 having the curved treatment portion 107 incorporated mechanical asymmetries that naturally have a tendency to include tip excursion in at least two, and possibly all three axes, x, y, and z of a three-dimensional right-handed coordinate system. If not balanced properly, excursions other than longitudinal will reflect a moment or force back to the transducer, causing inefficiencies and/or loss of lock to the longitudinal drive frequency, and possibly failure and/or fracture. For example, the curved treatment portion 107 may be described as having a positive curvature in the x-z plane. This curvature will cause excursions in at least both the x and z directions when activated.

It is possible to balance forces and/or moments caused by non-longitudinal tip excursion of a functional asymmetry, such as the curved treatment portion 107, by placing the center of mass of the curved treatment portion 107 about the center-line of the ultrasonic system in accordance with the present invention. It is desirable to balance a system below 15% non-longitudinal excursion proximal to the functional asymmetry, and it is preferable to balance below 5% non-longitudinal excursion proximal to the functional asymmetry. One method of locating the center of mass about the center line uses an anti-curve, such as the anti-curve 106.

A normalized non-longitudinal excursion percentage in an ultrasonic blade may be calculated by taking the magnitude of the excursion in the non-longitudinal direction, and dividing that magnitude by the magnitude of the maximum vibration excursion in the longitudinal direction (also called the primary vibration excursion), and then multiplying the dividend by one hundred. Primary tip vibration excursion is the magnitude of the major axis of the ellipse or ellipsoid created by a point on the distal most end, designated the terminal end, of curved treatment portion 107 when the ultrasonic surgical instrument 100 is activated. The primary tip vibration excursion and the primary vibration excursion may be equivalent or different, depending on the relationship between the longitudinal motion direction and the direction of the major axis of the ellipse or ellipsoid.

FIGS. 2 and 3 illustrate a cross-section plane 113, normal to the tangent of the longitudinal axis of the curved treatment portion 107, in which the blade 152 is symmetric about both the vertical and horizontal axes in the illustrated embodiment. The cross section of the curved treatment portion 107 at the cross-section plane 113 is illustrated as substantially rectangular, with dimensions about 0.057 inches height by about 0.085 inches width. In some alternate embodiments, the cross section of the curved treatment portion 107 at the cross-section plane 113 may be about 0.016λ height by about 0.024λ width. The curved treatment portion 107 is illustrated as about 0.545 inches to about 0.572 inches in length, and about 0.156λ to about 0.164λ in some alternate embodiments.

FIG. 3 illustrates a tip deflection 109 of about 0.070 inches of the edge of the curved treatment portion 107 relative to the center line 104. In some alternate embodiments the tip deflection 109 may be about 0.020λ, for example. A curve deflection 110 of about 0.040 inches of the bottom of the curved treatment portion 107 relative to the center line 104 is also illustrated. In some alternate embodiments the curve deflection 110 may be about 0.011λ, for example. A curve depth 111 of about 0.060 inches of the top of the curved treatment portion 107 relative to the center line 104 is also illustrated. In some alternate embodiments the curve depth 111 may be about 0.018λ, for example.

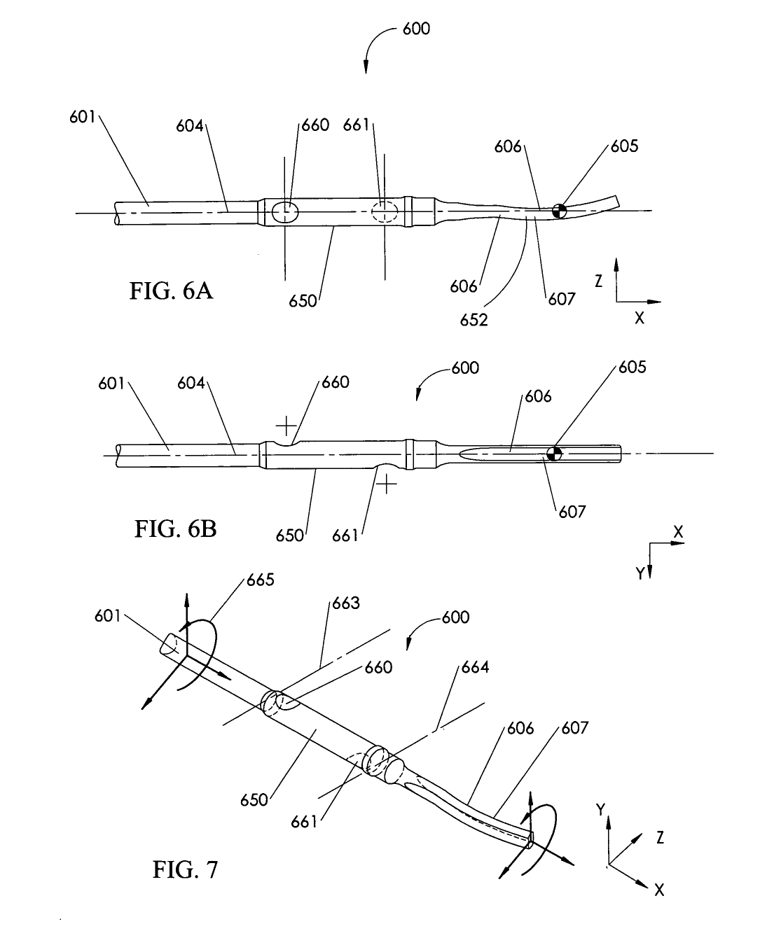

FIGS. 6 through 7 illustrate a blade 600 combining features of FIG. 1 and features of FIG. 4 as described in prior art. The figures illustrate the three dimensional positioning of the center of mass 605 relative to the central axis 604. In the particular example illustrated in FIGS. 6 through 7, the anti-curve 606 is illustrated as angling the curved treatment portion 607 about 6 degrees to about 12 degrees, and more particularly about 8.13 degrees, to position the center of mass 105 about the central axis 604, thereby reducing undesired transverse motion in the waveguide 650. Notches 660 and 661, positioned opposite each other with respect to the central axis 604 and whose axis 663 and 664 are parallel to the Z-axis, induce lateral motion in cutting blade 607. The combination of the lateral motion and the longitudinal motion of the blade 607 results in an undesired torsional motion 665 to the waveguide 601.

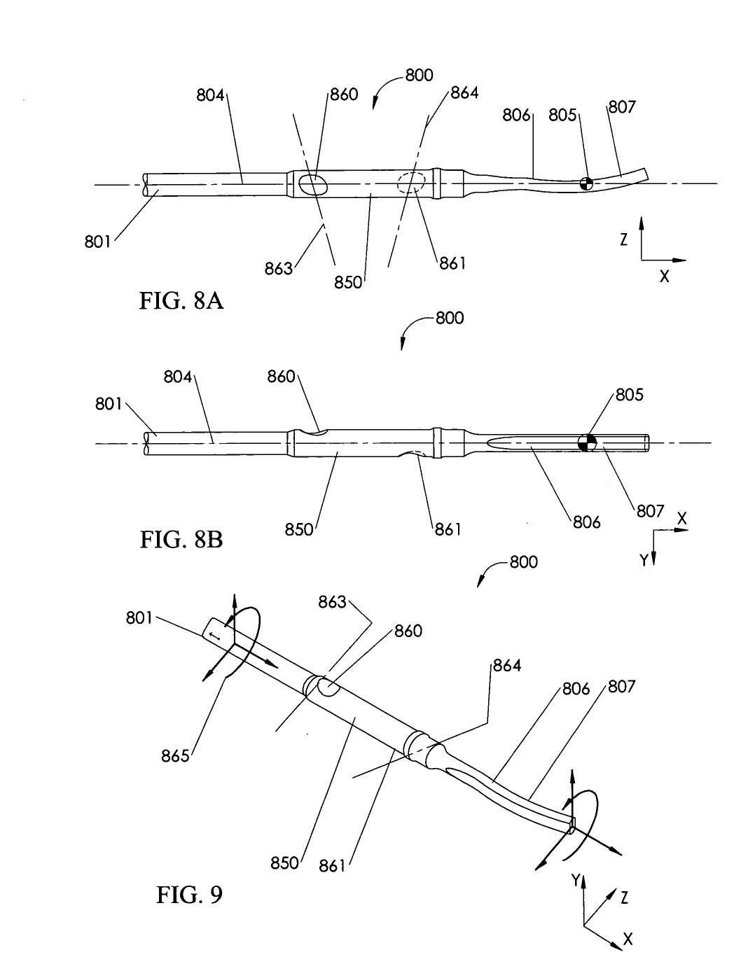

FIGS. 8 through 9 illustrate a blade 800 combining features of FIG. 1 and asymmetrical notches configured to produce asymmetrical motion in the end effector and reduce axial torsion motion. The figures illustrate the three dimensional positioning of the center of mass 805 relative to the central axis 804. In the particular example illustrated in FIGS. 8 through 9, the anti-curve 806 is illustrated as angling the curved treatment portion 807 about 6 degrees to about 12 degrees, and more particularly about 8.13 degrees, to position the center of mass 805 about the central axis 804, thereby reducing undesired transverse motion in the waveguide 850. Notches 860 and 861, positioned opposite each other with respect to the central axis 804 and whose axis 863 and 864 are substantially non-parallel to the Z-axis, induce lateral motion in cutting blade 807. The non-parallel or skewed configuration of the axis of the notches results in a significantly-reduced torsional motion 865 to the waveguide 801.

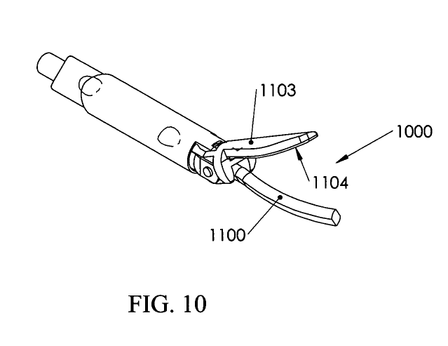

The end-effector 1000 illustrated in FIG. 10 includes the blade 1100 that is configured to operate accordance with the present invention and in clamping cooperation with a clamp arm. The clamp arm 1103 includes a clamp pad 1104 configured to apply pressure against the blade 1100 in order to cut and/or coagulate tissue disposed between the clamp arm 1103 and the blade 1100.

Example embodiments illustrated herein include mass balancing in accordance with the present invention. Typically, symmetrical mass balance may be implemented using symmetrical cross-sections of waveguide and blade portions, thereby reducing the amount of imbalance in an ultrasonic surgical instrument. Curved blade shapes in accordance with the present invention include their center of mass centered about the central axis of the blade's waveguide. An anti-curve proximal to curve of the blade may be used to position the blade's center of mass about the waveguides central axis, thereby reducing undesirable transverse motion in the waveguide.

Curved blade portions provide for an out and around surgical technique, and allow for passage of the blade through a trocar. Embodiments of blades in accordance with the present invention may include a flat front surface that may be used as a coagulating surface. The flat front surface may alternately be modified as a cutting surface. Curved blades used in clamping instruments may incorporate non-parallel motion with respect to their clamp pad, aiding in cutting and coagulation.

The dimensions shown in the figures and the above text are for purposes of illustration and not of limitation. For example, dimensions may vary, typically up to 35% from the designated numbers, without departing from the scope of the present invention. Dimensions may be given as multiples of the wavelength λ, for example, Ti6Al4V titanium alloy may have a ½ wavelength λ=1.74 inches at 55.5 kHz. It is understood that varying a dimension of an element may require altering other dimensions in order to maintain balance in accordance with the present invention.

Each feature disclosed in this specification (including any accompanying claims, abstract, and drawings), may be replaced by alternative features having the same, equivalent or similar purpose, unless expressly stated otherwise. Thus, unless expressly stated otherwise, each feature disclosed is one example only of a generic series of equivalent or similar features.

While embodiments of the present invention have been shown and described herein, it will be obvious to those skilled in the art that such embodiments are provided as examples only. Numerous variations, changes, and substitutions will be apparent to those skilled in the art without departing from the invention. Accordingly, it is intended that the invention be limited only by the scope of the appended claims.

What is claimed is:

1. An ultrasonic blade, comprising:
a waveguide configured to transmit ultrasonic energy therethrough;
an end-effector provided at the distal end of the waveguide, the end-effector comprising a functional asymmetry; and
means for balancing the transverse excursion in the waveguide proximal to the end-effector, and
means for introducing asymmetrical motion in the distal portion of the waveguide, and
means for balancing torsion excursion in the waveguide proximal to the end-effector.

2. The blade of claim 1, wherein the functional asymmetry comprises a curve having a positive curvature, and wherein the transverse balancing means comprises an anti-curve having a negative curvature.

3. The blade of claim 2, wherein the torsion balancing means comprises an asymmetrical balance feature.

4. The blade of claim 1, wherein the transverse balancing means comprises a plurality of asymmetrical balance features.

5. The blade of claim 2, wherein the torsion balancing means comprises an asymmetrical balance feature.

6. An ultrasonic surgical device, comprising:
an elongated waveguide configured to transmit ultrasonic energy therethrough, the elongated waveguide comprising a center-line extending through the center of mass of the elongated waveguide;
an end-effector provided at the distal end of the waveguide, the end-effector comprising a curved portion having a positive curvature, the positive curvature of the curved portion producing an offset of the center of mass of the curved portion; and
an anti-curved portion positioned between the elongated waveguide and the curved portion, the anti-curve having a negative curvature, the negative curvature configured to correct the offset of the center of mass of the curved portion, thereby substantially balancing the ultrasonic surgical device; and
a first notch positioned proximal to the end-effector, the first notch axis at an acute angle to the center-line; and
a second notch positioned proximal to the first notch.

7. A blade of claim 6, wherein the positive curvature defines a curve plane; and
the first notch axis is not perpendicular to the curve plane.

8. The device of claim 6, comprising a clamp arm configured to opposably clamp against the curved portion, the clamp arm actuatably movable from an open position to a clamped position.
